(19) United States
(12) Patent Application Publication
(10) Pub. No.: US 2011/0207952 A1
(43) Pub. Date: Aug. 25, 2011

(54) CHOLESTEROL EXTRACTION FROM ALGAE AND PREPARATION OF VEGAN VITAMIN D3

(76) Inventor: RAFAEL AVILA, Farmingdale, NY (US)

(21) Appl. No.: 13/027,847

(22) Filed: Feb. 15, 2011

Related U.S. Application Data
(60) Provisional application No. 61/306,600, filed on Feb. 22, 2010.

(51) Int. Cl. C07J 75/00; C07C 401/00; B01J 19/08

(52) U.S. Cl. 552/545; 552/653; 204/157.6

(57) ABSTRACT

A process for producing vegan vitamin D3 from an algal waste processing stream by saponifying the waste stream to separate out and removing unsaponifiable cholesterol and converting it to vitamin D3 by ultraviolet irradiation, while precipitating out the saponified fatty acids, triglycerides and polysaccharides.

### CROSS-REFERENCE TO RELATED APPLICATION

This application claims the benefit of U.S. Provisional Application Ser. No. 61/306,600, filed Feb. 22, 2010, the disclosure of which is incorporated herein by reference.

### FIELD OF THE INVENTION

This invention relates to a method of recovering cholesterol from either algae or from an algal waste stream generated during the processing of marine polymers, antioxidants, carotenoids, polysaccharides, fibers algae and, more specifically, to the recovery of vegan cholesterol from either algae or from the waste stream generated in the processing of algae into, for example, carageenan, antioxidants, carotenoids, etc. and to the manufacture of vegan cholcalciferol (vitamin D3) from the vegan cholesterol so-obtained.

### BACKGROUND OF THE INVENTION

The lack of sufficient vitamin D3 in our diets is being brought to our attention on a daily basis by the media in recounting warnings from physicians and nutritionists on the consequences of such inadequacy, namely, osteoporosis in the elderly, a deficient immune system and disorders of the endocrine glands.

In the current manufacture of vitamin D3, also known as cholcalciferol, cholesterol of animal origin is subjected to a dehydrohalogenation reaction to convert it to 7-dehydrocholesterol and then to cholcalciferol by ultraviolet irradiation. If the cholesterol source is fish, it raises issues with regard to environmental impact, endangered species, and mercury and pesticide accumulation. If the cholesterol source is from livestock tallow, it too, raises environmental concerns, and also the need for veterinary certificates, religious issues with regard to kosher and halal status, mad cow disease and swine flu, among others. The foregoing problems also arise with cholesterol derived from lanolin.

While the health food and the nutritional supplement industry has long had a desire to develop a vegetarian vitamin D3 derived exclusively from plant or non-animal sources, which would avoid the problems and concerns identified with vitamin D3 made from fish, lanolin or livestock-derived cholesterol, currently, there is no availability of such a plant-derived or vegan vitamin D3.

### SUMMARY OF THE INVENTION

It has been found that by separating and recovering the mixture of sterols present in algae, vegan cholesterol can be effectively separated and recovered therefrom and converted to yield vegan cholcalciferol.

It has also been found that in the commercial processing of algae into various marine polymers, notably carageenan, agar and alginates, that the waste stream from such processing, includes a significant percentage of sterols, which when properly treated and processed can result in the separation and recovery of vegan cholesterol, which can then be used in the manufacture of vegan vitamin D3.

The commercial processing of algae in the manufacture of marine polymers, the extracting of oils for biofuels, fucoidans as dietary antioxidants, alginates and alginic acid are some of the important processes used in many countries of the world which result in a multiplicity of waste streams which have not been exploited to retrieve the cholesterol values to be found therein.

It is an object of the present invention to provide a process for the preparation of vegan cholesterol from algae.

It is another object of the present invention to provide a process for the removal and recovery of vegan cholesterol from algal waste processing streams.

It is another object of the present invention to prepare vegan cholesterol and thereafter vegan vitamin D3 therefrom which is economically viable and competitive with fish-based and livestock-based vitamin D3.

### DETAILED DESCRIPTION OF THE INVENTION

It has been found that by subjecting algae to processes for extracting and separating the mixture of sterols found therein from non-sterol constituents or by saponifying algal waste streams or subjecting them to alternative separation methods, vegan cholesterol can be successfully isolated and recovered from the naturally occurring sterols found therein and can be converted to 7-dehydrocholesterol by dehydrohalogenation, and thereafter to vitamin D3 by ultraviolet irradiation.

In the industrial processing of algae to manufacture carageenan an alkali processing step is employed initially to extract the carageenans, which is a valuable marine polymer, and to remove the fatty acids, polysaccharides, sterols and other non-polymer, non-carageenan materials, collectively referred to as the waste from the process. Applicant has found that the sterols, which are part of the waste stream often, contain a majority of cholesterol as compared to other sterols. Waste streams from the processing of some algal species, such as Irish moss, contain sterol fractions that are about 95% by weight cholesterol and 7-dehydrocholesterol and can be recovered, successfully processed and used in the manufacture of a wholly-vegan vitamin D3.

Since there are literally thousands of species of algae ranging from unicellular microscopic organisms (microalgae) to multicellular organisms of great size (macroalgae), algae are, to say the least, highly diverse as to form and size. The industrial uses of algae in North America and Europe have typically been as a raw material from which to extract the marine polymers agar and carageenans from red algae and to extract alginates from brown algae.

There are several carageenans, which differ in their chemical structure and properties, and therefore in their ultimate uses. The carageenans which are of commercial interest are called iota, kappa and lambda.

Their uses are related to their ability to form thick solutions or gels, and they vary as follows:

| | |
|---|---|
| Iota | Elastic gels formed with calcium salts. |
| | Clear gel with no bleeding of liquid (no |
| | synaeresis). |
| | Gel is freeze/thaw stable. |
| Kappa | Strong, rigid gel, formed with potassium |
| | salts. |
| | Brittle gel forms with calcium salts. |
| | Slightly opaque gel, becomes clear with |
| | sugar addition. |
| | Some synaeresis. |
| Lambda | No gel formation, forms high viscosity |
| | solutions. |

The carageenan composition in red seaweeds differs from one species to another.

| | |
|---|---|
| Chondrus crispus | mixture of kappa and lambda. |
| Kappaphycus alvarezii | mainly kappa. |
| Eucheuma denticulatum | mainly iota |
| Gigartina skottsbergii | mainly kappa, some lambda. |
| Sarcothalia crispata | mixture of kappa and lambda. |

Refined carageenan is now sometimes referred to as filtered carageenan. It was first made from Chondrus crispus, but the current process for making carageenan can use all of the five (5) species of red algae or seaweed identified above.

The seaweed is washed initially to remove sand, salts and other foreign matter. It is then heated with water containing an alkali, such as sodium hydroxide, for several hours, with the time depending on the seaweeds being extracted and determined by prior small-scale trials, or experience. Alkali is used because it causes a chemical change that leads to increased gel strength in the final product. In chemical terms, it removes some of the sulfate groups from the molecules and increases the formation of 3,6-AG: the more of the latter, the better the gel strength.

The seaweed that does not dissolve is removed by centrifugation or a coarse filtration, or a combination of both. The solution is then filtered again, in a pressure filter using a filter aid that helps to prevent the filter cloth becoming blocked by fine, gelatinous particles. At this stage, the solution contains 1-2 percent carageenan and this is usually concentrated to 2-3 percent by vacuum distillation and ultrafiltration.

The processor now has a clear solution of carageenan and there are two methods for recovering it as a solid. An alcohol-precipitation method can be used for any of the carrageenans. A gel method can be used for kappa-carageenan only, and the gel can be dehydrated either by squeezing or by subjecting it to a freeze-thaw process.

Since the major constituents of the algal waste stream after the removal of marine polymers are polysaccharides, triglycerides, fatty acids and sterols, Applicant has determined that the separation and recovery of cholesterol and/or 7-dehydrocholesterol, from which cholcalciferol (also known as vitamin D3), can be manufactured simply and cheaply, would represent a significant value-added treatment for algal waste streams. This is especially so since, for some species, about 95% by weight of the sterols present in red algae are cholesterol per se or naturally occurring derivatives thereof.

Such a high cholesterol ratio relative to the total sterols present, namely, about 20:1, permits the use of simple methods of separation, rather than undertaking the difficult and expensive task of separating cholesterol from other sterols. A simple and inexpensive saponification with sodium or potassium hydroxide serves to separate the sterols from the fatty acids and triglycerides. Fatty acids and triglycerides will saponify, while sterols will not. Thus, subsequent to saponification, separation is essentially a straightforward operation since the saponified fatty acids and triglycerides have a greater density than the sterols, resulting in the fatty acids and triglycerides precipitating, while the sterols, which are 95% cholesterol, will float on the surface of the alkaline solution and can be removed by, for example, a mechanical skimming or cone separation operation.

Alternative methods of separation in view of the high cholesterol ratio relative to the total sterol which can be employed with algae or with an algal waste stream include distillation, cone separation, centrifugation, filtration, high pressure liquid chromatography, freeze separation and film separation.

Since alkalization is the major step in freeing the marine polymer carageenan from the algae, the adding of additional alkali to the waste stream is effective in initiating the saponification process, and providing a relatively easy and inexpensive operation to effectively complete the full separation process.

The cholesterol is then subjected to the chemical process of conversion to 7-dehydrocholesterol using any number of dehydrohalogenation processes or other processes known to those skilled in the art, for example, the process disclosed in U.S. Pat. No. 2,542,291. The 7-dehydrocholesterol is then irradiated with ultraviolet light of a suitable frequency to form cholcalciferol, which is also known as vitamin D3. Thus, vegan-cholesterol and vegan vitamin D3 are derived as valuable by-products from the algal waste processing stream by the process of the present invention. Heretofore, the traditional by-product was a low-value added product used for livestock feed. The availability of a wholly vegan form of vitamin D3 in the marketplace will likely induce many individuals to begin taking this important vitamin, who previously were reluctant to take animal derived vitamin D3 for health or religious reasons.

While the conversion of cholesterol to 7-dehydrocholesterol is described as occurring after the separation of the sterols from the waste stream, it is to be understood that the dehydrohalogenation reaction can also take place before the separation of the sterols from the waste stream.

The various separations and conversions previously disclosed need not necessarily occur in the sequence in which they have been described. The steps can be rearranged. As an example, the processes for the conversion of cholesterol to 7-dehydrocholesterol and the process of irradiating 7-dehydrocholesterol to form cholcalciferol may be carried out in the so-called “dirty” unseparated mix of waste stream materials (or fraction thereof), saving the separation of calciferol-sterols (primarily cholcalciferol) from other fatty acids for the last step.

It will be understood that various changes in the details that have been described herein in order to explain the nature of the present invention may be made by those skilled in the art without departing from the principle and scope of the invention as expressed in the appended claims.

1. A process for recovering cholesterol and/or 7-dehydrocholesterol from an algal processing waste stream, which comprises:
saponifying an algal processing waste stream, which includes sterols and fatty acids concurrently with or subsequent to extracting at least one marine polymer from said stream, to recover the unsaponified sterols and precipitating the saponified fatty acids thus yielding a mixture of sterols which is predominantly cholesterol and/or 7-dehydrocholesterol from the sterols.

2. The process of claim 1, wherein the marine polymers is carageenan.

3. The process of claim 1, wherein the alga, from which the waste stream is derived includes at least one member selected from the group consisting of red, green, blue and brown algae and mixtures thereof.

4. The process of claim 1, wherein the cholesterol is converted by a dehydrohalogenation reaction to form 7-dehydrocholesterol.

5. The process of claim 4, wherein the 7-dehydrocholesterol is irradiated with ultraviolet light to form cholcalciferol (vitamin D3).

6. A vegan cholcalciferol (vitamin D3) prepared in accordance with the process of claim 5.

7. A process for preparing vegan vitamin D3, which comprises:
saponifying an algal processing waste stream, which includes sterols and fatty acids, subsequent to the extraction of carageenans, in order to recover the unsaponified sterols and precipitating the saponified fatty acids, thus yielding a mixture of sterols which is predominantly vegan cholesterol and vegan 7-dehydrocholesterol;
converting the vegan cholesterol to vegan 7-dehydrocholesterol by dehydrohalogenation; and
irradiating the vegan 7-dehydrocholesterol with ultraviolet light to convert it to vegan vitamin D3.

8. A process for recovering cholesterol and/or 7-dehydrocholesterol from an algal processing waste stream, which comprises:
subjecting an algal processing waste stream which includes a mixture of sterols, marine polymers, fatty acids, marine fibers, marine antioxidants and marine carotenoids to a sterol separation process selected from the group consisting of distillation, cone separation, column separation, centrifugation, high pressure liquid chromatography, filtration, freeze-thaw separation and film separation concurrently with or subsequent to extracting members selected from the group consisting of marine polymers, marine fibers, marine polysaccharides and marine carotenoids, to separate and recover the mixture of sterols which are predominantly cholesterol and/or 7-dehydrocholesterol while removing the fatty acids.

9. The process of claim 8, wherein the cholesterol is converted by a dehydrohalogenation reaction to 7-dehydrocholesterol.

10. The process of claim 9, wherein the 7-dehydrocholesterol is irradiated with ultraviolet light to cholcalciferol (vitamin D3).

11. A vegan cholcalciferol (vitamin D3) prepared in accordance with the process of claim 10.

12. A process for recovering cholesterol from algae, which comprises:
subjecting algae which includes a mixture of sterols and non-sterol constituents to a sterol separation process to separate and recover the mixture of sterols which are predominantly cholesterol and/or 7-dehydrocholesterol while removing the non-sterol constituents.

13. The process of claim 12 wherein the sterol separation process is selected from the group consisting of distillation, cone separation, column separation, centrifugation, high pressure liquid chromatography, filtration, freeze-thaw separation and film separation and saponification.

14. The process of claim 13, wherein the cholesterol is converted by a dehydrohalogenation reaction to 7-dehydrocholesterol.

15. The process of claim 14, wherein the 7-dehydrocholesterol is irradiated with ultraviolet light to form cholcalciferol (vitamin D3).

16. A vegan cholcalciferol (vitamin D3) prepared in accordance with claim 15.
